(19) United States
(12) Patent Application Publication
(10) Pub. No.: US 2017/0119824 A1
(43) Pub. Date: May 4, 2017

(54) A METHOD FOR THE TREATMENT OF GLIOBLASTOMA WITH WHARTON JELLY-MESENCHYMAL STEM CELLS (WJ-MSC) DERIVED FROM HUMAN UMBILICAL CORD

(71) Applicant: Neelam Krishnan Venkataramanaa, Bangalore (IN)

(72) Inventor: NEELAM KRISHNAN VENKATARAMANAA, Bangalore (IN)

(21) Appl. No.: 15/319,761

(22) PCT Filed: May 20, 2015
(86) PCT No.: PCT/IN2015/000214

§ 371 Date: Dec. 17, 2016

(30) Foreign Application Priority Data
May 24, 2014 (IN) 1535/CHE/2014

(51) Int. Cl. A61K 35/28; C12N 5/0775; A01N 1/02

(52) U.S. Cl. CPC A61K 35/28; A01N 1/0226; C12N 5/0668

(57) ABSTRACT

The embodiments of the present invention provide a method for the treatment of glioblastoma using Wharton Jelly-mesenehmal stem cells (WJ-MSC) derived from human umbilical cord. The mesenchymal stem cells have potential to inhibit the glioblastoma cancer cells. For isolating the MSC, the donor is screened for infectious diseases. The consent from the donor is taken for the collection of the umbilical cord sample. The MSC are mechanically harvested or isolated from the Wharton's jelly of umbilical cord. The WJ-MSC are cultured and propagated in vitro and harvested. The harvested WJ-MSC is subjected for characterization. The characterized WJ-MSC's are cryo-preserved. The tumors which do not respond to temozolomide (TMZ) respond to WJ-MSC. WJ-MSC does not exert any toxic effect on any human organ. WJ-MSC are cryoprotective to healthy cells and cytotoxic to glioblastoma cells.

### CROSS-REFERENCE TO RELATED APPLICATIONS

The application is a National Phase Application filed with respect to the PCT Application No. PCT/IN2015/000214 filed on May 20, 2015 with the title “A METHOD FOR TARGETING GLIOBLASTOMA WITH WHARTON JELLY-MESENCHYMAL STEM CELLS (WJ-MSC) DERIVED FROM HUMAN UMBILICAL CORD”, The application further claims the priority of the Indian Provisional Patent Application with No. 1535/CHE/2014 filed on Mar. 24, 2014 and post dated by 2 months to May 24, 2014 the title “A METHOD FOR THE TREATMENT OF GLIOBLASTOMA WITH WHARTON JELLY-MESENCHYMAL STEM CELLS (WJ-MSC) DERIVED FROM HUMAN UMBILICAL CORD”. The contents of the above mentioned applications are incorporated in its entirety by reference herein.

### BACKGROUND

### TECHNICAL FIELD

The present invention generally relates to the field of stern cells. The present invention particularly relates to the treatment of cancer using stem cells. The present invention more particularly relates to the isolation Wharton jelly-mesenchyinal stem cells (WJ-MSC) from human umbilical cord derived for the treatment of glioblastoma.

### DESCRIPTION OF THE RELATED ART

Cancer is medically known as malignant neoplasm, which involves unregulated cell growth. The cancer cells divide and grow uncontrollably, thereby forming malignant tumors and invading the nearby parts/organs of the human body. The cancer may also spread to the more distant parts of the human body through the lymphatic system or blood stream. Not all tumors are cancerous. The benign tumors do not invade the neighboring tissues and do not spread throughout the body.

The causes of cancer are diverse, complex and are partially understood. Several factors are known to increase the risk of cancer and the factors include but not limited to the use of tobacco, dietary factors, certain infections (especially viral infections) exposure to radiation, lack of physical activity, obesity, environmental pollutants and genetic factors/mutations.

The most commonly known cancers are blood cancer, bone cancer, brain cancer or brain tumor, breast cancer, digestive/gastrointestinal cancer, endocrine cancer, eye cancer, genitourinary cancer, gynecologic cancer, head and neck cancer, respiratory cancer, skin cancer etc.

Cancer is detected in a number of ways, including the presence of certain signs and symptoms, screening tests and medical imaging. Generally microscopic examination of tissue sample or in vitro culture reveals the presence of cancer. Cancer is usually treated by chemotherapy, radiation therapy and surgery. The chances of surviving the cancer vary greatly based on the type and location of the cancer and the extent of cancer growth at the initial stage of the treatment.

Glioblastoma multiforme (GBM) or “glioblastoma” is the most common and most aggressive malignant primary brain tumor in humans involving glial cells. Glioblastoma a accounts for 52% of the damage of all functional tissues and 20% of all intracranial tumors. The common symptoms of glioblastoma include seizure, vomiting, headache and Neurological deficits. The symptoms also include progressive memory loss, personality or neurological deficit due to temporal and frontal lobe involvement.

The treatment of glioblastoma is very difficult due to several complicating factors such as the tumor cells are very resistant to conventional therapies; the brain is susceptible to damage due to conventional therapy; the brain has a very limited capacity to repair itself; and many drugs cannot cross the blood-brain barrier to act on the tumor.

The treatment of glioblastoma based on symptomatic therapy focuses on the symptoms and improving the patient's neurological function. The primary supportive agents or therapeutic agents are anticonvulsants and cortico-steroids. This therapy is not effective to treat glioblastoma as it only focuses on relieving the symptoms and not alleviating the cause for the symptoms.

The palliative treatment is usually conducted to improve a quality of life and to achieve a longer survival period. The palliative treatment includes surgery, radiation therapy and chemotherapy.

Surgery is the most common method for the treatment of glioblastoma or brain tumor. Surgery is the first stage of treatment for treating glioblastoma. Surgery is used to take a section of tumor for a pathological diagnosis and also to remove the larger tumor mass pressing against the brain before administering the secondary treatments such as radiotherapy and chemotherapy. This also reduces the intra cranial pressure and the tumor load. However it is not possible to achieve maximum cyto-reduction in eloquent areas of the brain. The surgery in combination with chemotherapy and radiotherapy prolong the survival of the patient. Glioblastoma cells are widely infiltrative through the brain at diagnosis. In spite of a total surgical removal of entire tumor mass, the “satellite lesions” appear near the original site or at more distant sites within the brain. Other modalities including radiation are used after surgery in an effort to suppress and slow the recurrence of the disease. The surgical method for the treatment of glioblastoma is inefficient as the tumors develop recurrently.

After the surgery the patients are subjected to radiotherapy. The radiotherapy is the mainstay of the treatment for people with glioblastoma. Radiotherapy after surgery can reduce the size of tumor. Whole brain radiotherapy does not show improvement in patients, when compared to the more precise and targeted 3D conformal radiotherapy. Radiation therapy has many side effects such as skin color change, dry skin, fatigue, diarrhea, hair loss, mouth ulcer, nausea/vomiting, swelling, impotency etc.

In addition to the radiotherapy, chemotherapy (mainly includes temozolamide) improves the patient's survival chance. Most studies have shown that there is less benefit from the chemotherapy as chemotherapy has several side effects. The drug temozolamide (TMZ) not only kills the tumor cells but also kills the surrounding healthy cells. TMZ affects the physiological functioning of all the organs. As a result, the patient is affected medically and emotionally. Further chemotherapy has a number of side effects which include nausea and vomiting, alopecia (hair loss), fatigue, hearing impairment (deafness. ototoxicity, neutropenia, thrombocytopenia, anemia, mucositis, loss of appetite, dryness and embrittlement of skin and nails, cognitive problems, infertility, bowel movement problems, depression etc.

Stem cells are undifferentiated biological cells that can be differentiate into specialized cells and can be divided (through mitosis) to produce more stem cells. They are found in multicellular organisms. In mammals, there are two broad types of stem cells called “embryonic stem cells”, which are isolated from the inner cell mass of blastocysts, and “adult stem cells”, which are found in various tissues. The Wharton's jelly of the umbilical cord contains mucoid connective tissue and fibroblast-like cells. Mesenchymal stem cells in Wharton's jelly express several stem cell genes, including telomerase. Wharton jelly mesenchymal stem cells (WJ-MSC) are extracted, cultured, and induced to differentiate into manure cell types such as neurons. Wharton's jelly is therefore a potential source of adult stem cells. Mesenchymal stem cells, or MSCs, are multipotent stromal cells that can be differentiated into a variety of cell types, suchas osteoblasts (bone cells), chondrocytes (cartilage cells), and adipocytes (fat cells).

Wharton's jelly-mesenchymal stem cells (WJ-MSC) are emerging as most promising anti-cancer agents which have an enormous potential to be utilized to treat several types of cancer. MSC have inherent tumor-trophic migratory properties, which allows them to serve as vehicles/carriers for delivering effective, targeted therapy to the isolated tumors and metastatic disease. MSC have been readily engineered to express the anti-proliferative, pro-apoptotic, anti-angiogenic agents that specifically target the different types of cancers.

Hence, there is a need for a method for the treatment of glioblastoma without any side effects. Also there is a need for a method for the treatment of glioblastoma utilizine Wharton's jelly-mesenchymal stem cells (WJ-MSC).

The above mentioned shortcomings, disadvantages and problems are addressed herein and which will be understood by reading and studying the following specification.

### OBJECTIVES OF THE EMBODIMENTS

The primary objective of the embodiments herein is to provide a method for the treatment of ghoblastoma by using Wharton's jelly-mesenclaymal stem cells (WJ-MSC).

Another objective of the embodiments herein is to provide a method for the treatment of glioblastoma without any side effects.

Yet another objective of the embodiments herein is to provide a method for the treatment of glioblastonia involving Wharton's jelly-mesenchymal stem cells (WJ-MSC) which does not lead to brain damage.

Yet another objective of the embodiments herein is to provide a method for the treatment of glioblastoma involving Wharton's jelly-mesenchymal stem cells (WJ-MSC), for delivering the stem cells to treat the tumor independent of blood brain barrier.

Yet another objective of the embodiments herein is to provide a method for the treatment of glioblastoma using Wharton's jelly-mesenchymal stem cells (WJ-MSC) thereby not only treating the symptoms but also improving the patient's neurological functions and eradicating the tumor.

Yet another objective of the embodiments herein is to provide a method for the treatment of glioblastoma independent of surgery, radiation therapy and chemotherapy techniques.

Yet another objective of the embodiments herein is to provide a simple method for the isolation of mesenchymal stem cells (MSC) from Wharton's Jelly (WJ).

Yet another objective of the embodiments herein is to provide a simple mechanical method for the isolation of Wharton's jelly-mesenchymal stem cells (WJ-MSC) from umbilical cord.

Yet another objective of the embodiments herein is to provide a simple method for administration of the Wharton's jelly-mesenchymal stem cells (WJ-MSC) into glioblastoma patients.

These and other objects and advantages of the embodiments will become readily apparent from the following detailed description taken in conjunction with the accompanying drawings.

### SUMMARY

The various embodiments herein provide a method for the treatment of glioblastoma by using Wharton jelly-mesenchymal stem cells (WJ-MSC) derived from human umbilical cords. The WJ-MSC provides a method for the treatment of glioblastoma without any side effects. The method of glioblastoma treatment with the application of WJ-MSC is independent of blood brain barrier for the delivery of stem cells and to treat the tumor. The treatment method not only treats the carcinogenic symptoms but also improves the patient's neurological functions and eradicate tumor.

According to one embodiment herein, the method for isolating mesenchymal stem cells from Wharton jelly of umbilical cord comprising the following steps. An umbilical cord is collected from a donor. Then the umbilical cord is processed and preserved. The preserved umbilical cord is used for isolating the mesenchymal stem cells from the Wharton jelly of umbilical cord. The isolated mesenchymal stem cells are analyzed for surface antigens. The mesenchymal stem cells isolated from the Wharton jelly of umbilical cord is cryopreserved.

According to one embodiment herein, the method of processing and preserving umbilical cord comprises the following steps. An informed consent is obtained from an umbilical cord donor and umbilical cord is collected from the donor after receiving the consent. After collecting the umbilical cord, all the instruments are wiped with alcohol in a biosafety hood, and the alcohol is 70% v/v isopropyl alcohol. The umibilical cord is cut into pieces and the umbilical cord pieces are rinsed with a saline solution. The umbilical cord pieces are rinsed to remove blood clots. The umbilical cord pieces are washed with a Dulbecco's phosphate buffer saline solution. The buffer saline solution comprises of an antibiotic, and the antibiotic is an antimycotic agent (1×). The umbilical cord pieces are sterilized with alcohol for 45 seconds. The alcohol is 70% v/v ethanol. The umbilical cord pieces are washed with a Dulbecco's phosphate buffer saline (DBPS) for four times. The umbilical cord pieces are washed in DPBS to remove traces of ethanol. The umbilical cord pieces are further cut into into smaller pieces of 2-3 cm length. The umbilical cord pieces are stored in a saline solution.

According to one embodiment herein, the method of isolating the mesenchymal stem cells from the Wharton jelly of umbilical cord comprises the following steps. In the first step the umbilical cord is placed in a sterile petridish. The petridish comprises 5 ml of a saline solution. The umbilical cord is slit to expose the Wharton's jelly and the umbilical cord is slit using a foresep and scalpel. The Wharton's jelly is collected in a sterile 50 ml centrifuge tube. The Wharton's jelly is centrifuged at 15000 rpm for 15 minutes. The supernatant is discarded and the pellet is washed with Dulbecco's Phosphate Buffer Saline (DBPS) to obtain the mesenchymal stem cells. The cells are strained using a cell strainer in a sterile 50 ml falcon tube. The pore size of cell strainer is 100 μm. The cells are centrifuged at 15000 rpm for 15 minutes. The supernatant is discarded and the pellet is dissolved in a fresh culture medium. The pellet comprises mesenchymal stem cells. The mesenchymal stem cells are counted using a Haemocytometer. The mesenchymal stem cells are seeded on one cell stack with a density of 10,000 cells per cm2. The cell stack comprises 100 ml of culture media supplemented with 1 ng/ml basic fibroblast growth factor (bFGF). The cell stack is gently rocked for an evenly distribution of the cells on a laboratory rocker. The cell stack is incubated at 37° C. with 5% CO2. The media is changed completely after 72 hours of mesenchymal stem cell isolation process. The spent culture medium is replaced with a fresh culture medium after every five days. Further the mesenchymal stem cells are cultured for 15-20 days until a continency of 80%-90% is obtained.

According to one embodiment herein, the isolated mesenclrymal stem cells are analyzed for antigens. The isolated mesenchymal stem cells are tested positive for CD 73, CD 90 and CD 166 antigens. The mesenchymal stem cells are tested negative for CD34 and CD48 antigens, 95% of the mesenchymal stem cells are tested positive for CD 73, CD 90 and CD 166.

According to one embodiment herein, the method of targeting glioblastoma with Wharton's Jelly mesenchymal stem cells (WJ-MSCs) derived from human umbilical cord, comprises the following steps. The Wharton's Jelly mesenchymal stern cells (WJ-MSCs) are administered to the glioblastoma tumour. The effect of Wharton's Jelly mesenchymal stem cells (WJ-MSCs) on glioblastoma tumour is analyzed.

According to one embodiment herein, the Wharton's Jelly mesenchymal stem cells (WJ-MSCs) are injected to the glioblastoma tumour in the laboratory conditions.

According to one embodiment herein, the Wharton's Jelly mesenchymal stem cells (WJ-MSCs) stop the growth of glioblastoma tumour cells and induces programmed cell death (apoptosis).

According to one embodiment herein, the Wharton's Jelly mesenchymal stem cells (WJ-MSCs) reduce the proliferating glioblastoma tumour cell and reduce the uncontrollable growth of glioblastoma tumour cell.

According to one embodiment herein, the following steps are involved in the isolation and administration of the Wharton jelly-mesenchymal stem cells (WJ-MSC)to a glioblastoma patient. The consent from the umbilical cord donor is taken. The donor is screened for infectious disease. The sample is collected from the donor and the donor is informed of the collection of sample. A mechanical harvesting of Wharton's jelly-mesenchymal stem cells is done. In vitro culture and propagation of mesenchymal stem cellsis carried out. The stem cells are harvested. The characterization of the isolated, harvested mesenchymalstem cells is performed. The cryopreservation of the mesenchymal stem cells is carried out and the mesenchymal stem cells are administered to glioblastoma patient.

According to one embodiment herein, an umbilical cord is obtained for a prior isolation of the mesenchymal stem cells from the Wharton's Jelly. The umbilical cord donor is screened for infectious diseases such as human immune virus (HIV), Hepatitis B virus (HBV), Hepatitis C virus (HCV), Cytomegalovirus (CMV), and Venereal Disease Research Laboratorytest (VDRL test) as per international society for cellular therapy (ISCT) criteria. The consent from the umbilical cord donor is taken for the collection of umbilical cord sample and is informed of the collection. After the umbilical cord sample is obtained the mesenchymal stem cells are isolated from the Wharton's Jelly.

According to one embodiment herein, the isolation of mesenchymal stem cells from Wharton's jelly (WJ-MSC) is carried out by a simple mechanical method. The isolation of WJ-MSC is carried out in a cGMP compliant clean room. All the necessary instruments are brought into the biosafety hood and are wiped thoroughly with 70% isopropyl alcohol (IPA). A fresh human umbilical cord (normal delivery/cesarean section) obtained after birth is rinsed in the normal saline and the blood clots are removed. The umbilical cord is subjected to a sterile falcon tube containing Dulbecco's Phosphate Buffered Saline (DPBS). The umbilical cord is properly washed with DPBS containing antibiotic-antimycotic agent (1×) for three times. Again the umbilical cord is sterilized with 70% ethanol for 45 seconds. The umbilical cord pieces are washed with DPBS for 4 times to remove the traces of ethanol. The umbilical cord is cut into the pieces of 2-3 cms length and stored in a saline solution.

According to one embodiment herein, the mesenchymal stem cells are isolated and propagated by the following steps: a 5 ml of saline is taken in a sterile petridish and the umbilical cord piece is placed in the saline solution. A slit is made in the umbilical cord piece to expose the Wharton's jelly. The umbilical cord vessels are removed in the saline solution and the Wharton's jelly is removed using the sharp sterile forceps and a scalpel. The Wharton's jelly is collected in a sterile 50 ml centrifuge tube. The collected Wharton's jelly is centrifuged at 1500 rpm for 15 minutes. The supernatant is discarded and the pellet is washed once with Dulbecco's Phosphate Buffered Saline (DPBS). The cells are strained using the cell strainer (100 μm) in a sterile 50 ml falcon tube (around 45-50 ml). The cells are centlifuged at 1500 rpm for 15 minutes. The supernatant is discarded and the pellet is dissolved in a fresh culture medium. The cell count is done on using a Haemocytometer. The cells are seeded on one cell stack with a density of 10000 cells per cm². A 100 ml of culture media supplemented with 1 ng/ml basic fibroblast growth factor (bFGF) is added to one chamber cell stack. The cell stack is gently rocked for an even distribution of the cells and labeled as “Passage 0” (P-0), with ANSA code and date. The cell stack is incubated at 37° C. with 5% CO₂ and is observed every altemateday. A complete media change is done after 72 hours of isolation procedure. After every 5 days, the spent media is partially replaced with a fresh culture medium. The cells are cultured for 15-20 days until the confluency of 80%-90% is achieved in the flask.

According to one embodiment herein, each and every cell express a set of antigens. These set of antigen acts as a unique signature of a particular cell type. Immunophenotyping is a technique used to identify these surface marker/antigen expression. Once the set of antigens expressed by cells are identified a cell type is be defined example:- a blood cell, a muscle cell etc. Similarly stem cells are also defined and identified. Immunophenotyping of stem cells assures that the cells are stem cells and not other cells.

If a cell is defined as mesenchymal stem cell, then the cell should have CD73, CD90 and CD166 antigens. The cells should be positive for these antigens. Further the mesenchymal stem cells should be negative for CD34 and CD48 antigens. As these antigens are expressed by hematopoietic stem cells, the hematopoietic stem cells are not mesenchymal stem cells in nature. Further to call a population of cells as mesenchymal stromal cells, more than 95% of the cells should be positive for CD73, CD90 and CD166 antigen. Also the cells showing CD34 and CD 48 antigens should not exceed 2%.

According to one embodiment herein, the characterization of the isolated and harvested mesenchymal stem cells from Wharton's jelly is carried out. The characterization process includes identification of the morphology i.e. spindle shape of the cells; immunophenotype identification which includes CD34−ve, CD45−ve, CD73+ve, CD90+ve, CD166+ve testing; and tri-lineage differentiation potential testing which includes testing for adipocyte, osteocyte and chondrocyte differentiation.

According to one embodiment herein, the characterized Wharton's jelly derived mesenchymal stem cells (WJ-MSC) are cryopreserved.

According to one embodiment herein, the Wharton's jelly derived mesenchymal stem cells (WJ-MSC) are injected through the intravenous routeto the glioblastoma patient. In addition, when there is an opportunity for surgical intervention, these cells can be delivered directly into the tumor site. It is found that the tumors which do not respond to Temozolomide (TMZ) respond to WJ-MSC. Also WJ-MSC does not exert any toxic effect on any organ of the human body. WJ-MSC are cryoprotective to healthy cells and cytotoxic to glioblastoma cells.

According to one embodiment herein, Ocoprint® an explants tumor culture model to identify the anti-tumor effect of commonly used chemotherapeutics agents as well as Wharton's jelly derived mesenchymal stem cells on gliomas samples. In the laboratory, a tumor microenvironment similar to the body or physiological environment is created artificially in a dish. The tumor sample collected from patient is placed in this micro-environment in dish. Different anticancer agents including the mesenchymal stem cells derived from umbilical cord are applied on the tumor in the micro-environment. This mimics the application of these anti-cancer agents against the tumor when the tumor is in the human body. Simultaneously a control sample is maintained without any treatment or application of anti-cancer agents. After a specific treatment time or incubation the tumor samples are collected from the microenvironment and are subjected to a set of laboratory tests. The tests demonstrate the effect of anti-cancer agents on the tumor. The tests also reveal that the anti-cancer agents are able to kill tumor cells, reduce the number of tumor cells or limit the growth of tumor cells or the anti-cancer agent is converting the aggressively growing tumor cells to a programmed cell death mode.

The tests which are applied for testing effect of anti-cancer agents on the tumor are 1) Hematoxylin and eosin staining (H & E staining), 2) Ki67 is staining and 3) Caspase staining.

The H & E staining reveals the number of tumor cells present in the sample. The Ki67 staining indicates the growth pattern of tumor cells. The caspase staining shows that the aggressively growing cells turned into a programmed cell death mode or the dying cells.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPITION OF THE DRAWINGS

The other objects, features and advantages will occur to those skilled in the art from the following description of the preferred embodiment and the accompany drawings in which:

FIG. 1 illustrates a flow chart explaining a method for treating glioblastoma patients from human umbilical cord derived Wharton jelly-mesenchqual stem cells, according to one embodiment herein.

FIG. 2A-FIG 2C jointly illustrates a flow chart explaining a method for isolation and maintenance of Wharton jelly-mesenchymal stem cells from human umbilical cord sample, according to one embodiment herein.

FIG. 3 illustrates a graph indicating the immune-phenotype characterization of the Wharton jelly-mesenchymal stem cells, according to one embodiment herein.

FIG. 4 illustrates a micrograph indicating the shape of mesenchymal stem cells isolated and cultured from human Wharton's jelly sample, according to one embodiment herein.

FIG. 5 illustrates an Oncoprint® sensitivity report indicating the drug sensitivity test, according to one embodiment herein.

FIG. 6 illustrates a photograph indicating the effect of drugs on the cancer cell lines, according to one embodiment herein.

FIG. 7 illustrates a photograph indicating the effect of the mesenchymal stem cells on the tumors sample in vitro, according to one embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS

In the following detailed description, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a method for the treatment glioblastoma by using Wharton jelly-mesenchymal stem cells (WJ-MSC) derived from human umbilical cords. The WJ-MSC provides a method for the treatment of glioblastoma without any side effects. The method of glioblastoma treatment with the application of WJ-MSC is independent of blood brain barrier for the delivery of stem cells and to treat the tumor. The treatment method not only treats the tumor but also improves the patient's neurological functions, other symptoms and therefore the quality of life.

According to one embodiment herein, the method for isolating mesenchymal stem cells from Wharton jelly of umbilical cord comprising the following steps. An umbilical cord is collected from a donor. Then the umbilical cord is processed and preserved. The preserved umbilical cord is used for isolating the mesenchymal stem cells from the Wharton jelly of umbilical cord. The isolated mesenchymal stem cells is analyzed for surface antigens. The mesenchymal stem cells isolated from the Wharton jelly of umbilical cord is cryopreserved.

According to one embodiment herein, the method of processing and preserving umbilical cord comprises the following steps. An informed consent is obtained from an umbilical cord donor and umbilical cord is collected from the donor after receiving the consent. After collecting the umbilical cord, all the instruments are wiped with alcohol in a biosafety hood, and the alcohol is 70% v/v isopropyl alcohol. The umbilical cord is cut into pieces and the umbilical cord pieces are rinsed with a saline solution. The umbilical cord pieces are rinsed to remove blood clots. The umbilical cord pieces are washed with a Dulbecco's phosphate buffer saline solution. The buffer saline solution comprises of an antibiotic, and the antibiotic is an antimycotic agent (1×). The umbilical cord pieces are sterilized with alcohol for 45 seconds. The alcohol is 70% viv ethanol. The umbilical cord pieces are washed with a Dulbecco's phosphate buffer saline (DBPS) for four times. The umbilical cord pieces are washed in DPBS to remove traces of ethanol. The umbilical cord pieces are further cut into smaller pieces of 2-3 cm length. The umbilical cord pieces are stored in a saline solution.

According to one embodiment herein, the method of isolating the mesenchymal stem cells from the Wharton jelly of umbilical cord comprises the following steps. In the first step the umbilical cord is placed in a sterile petridish. The petridish comprises 5 ml of a saline solution. The umbilical cord is slit to expose the Wharton's jelly and the umbilical cord is slit using a foresep and scalpel. The Wharton's jelly is collected in a sterile 50 ml centrifuge tube. The Wharton's jelly is centrifuged at 15000 rpm for 15 minutes. The supernatant is discarded and the pellet is washed with Dulbecco's Phosphate Buffer Saline (DBPS) to obtain the mesenchymal stem cells. The cells are strained using a cell strainer in a sterile 50 ml falcon tube. The pore size of cell strainer is 100 μm. The cells are centrifuged at 15000 rpm for 15 minutes. The supernatant is discarded and the pellet is dissolved in a fresh culture medium. The pellet comprises mesenchymal stem cells. The mesenchymal stem cells are counted using a Haemocytometer. The mesenchymal stem cells are seeded on one cell stack with a density of 10,000 cells per cm2. The cell stack comprises 100 ml of culture media supplemented with 1 ng/ml basic fibroblast growth factor (bFGF). The cell stack is gently rocked for an evenly distribution of the cells on a laboratory rocker. The cell stack is incubated at 37° C. with 5% CO2. The media is changed completely after 72 hours of mesenchymal stem cell isolation process. The spent culture medium is replaced with a fresh culture medium after every five days. Further the mesenchymal stem cells are cultured for 15-20 days until a confluency of 80%-90% is obtained.

According to one embodiment herein, the isolated mesenchymal stem cells are analyzed for antigens. The isolated mesenchymal stem cells are tested positive for CD 73, CD 90 and CD 166 antigens. The mesenchymal stem cells are tested negative for CD34 and CD48 antigens. 95% of the mesenchymal stem cells are tested positive for CD 73, CD 90 and CD 166.

According to one embodiment herein, the method of targeting glioblastoma with Wharton's Jelly mesenchymal stem cells (WJ-MSCs) derived from human umbilical cord, comprises the following steps. The Wharton's Jelly mesenchymal stem cells (WJ-MSCs) are administered to the glioblastoma tumour. The effect of Wharton's Jelly mesenchymal stem cells (WJ-MSCs) on glioblastoma tumour is analyzed.

According to one embodiment herein, the Wharton's Jelly mesenchymal stem cells (WJ-MSCs) injected to the glioblastoma tumour in the laboratory conditions.

According to one embodiment herein, the Wharton's Jelly mesenchymal stem cells (WJ-MSCs) stop the growth of glioblastoma tumour cells and induces programmed cell death (apoptosis).

According to one embodiment herein, the Wharton's Jelly mesenchymal stem cells (WJ-MSCs) reduce the proliferating glioblastoma tumour cell and reduce the uncontrollable growth of glioblastoma tumour cell.

According to one embodiment herein, the following steps are involved in the isolation and administration of the Wharton jelly-mesenchymal stem cells (WJ-MSC) to a glioblastoma patient. The consent from the umbilical cord donor is taken. The donor is screened for infectious disease. The sample is collected from the donor and the donor is informed of the collection of sample. A mechanical harvesting of Wharton's jelly-mesenchymal stem cells is done and in vitro culture and propagation of mesenchymal stem cells is carried out. The stem cells are harvested. The characterization of the isolated, harvested mesenchymal stem cells is performed. The cryopreservation of the mesenchymal stem cells is carried out and the mesenchymal stem cells are administered to glioblastoma patient.

According to one embodiment herein, an umbilical cord is obtained for a prior isolation of the mesenchymal stem cells from the Wharton's Jelly. The umbilical cord donor is screened for infectious diseases such as human immune virus (HIV), Hepatitis B virus (HBV), Hepatitis C virus (HCV), Cytomegalovirus (CMV), and Venereal Disease Research Laboratory test (VDRL test) as per international society for cellular therapy (ISCT) criteria. The consent from the umbilical cord donor is taken for the collection of umbilical cord sample and is informed of the collection. After the umbilical cord sample is obtained the mesenchymal stem cells are isolated from the Wharton's Jelly.

According to one embodiment herein, the isolation of mesenchymal stem cells from Wharton's jelly (WJ-MSC) is carried out by a simple mechanical method. The isolation of WJ-MSC is carried out in a cGMP compliant clean room. All the necessary instruments are brought into the biosafety hood and are wiped thoroughly with 70% isopropyl alcohol (IPA). A fresh human umbilical cord (normal delivery/cesarean section) obtained after birth is rinsed in the normal saline and the blood clots are removed. The umbilical cord is subjected to a sterile falcon tube containing Dulbecco's Phosphate Buffered Saline (DPBS). The umbilical cord is properly washed with DPBS containing antibiotic-antimycotic agent (1×) for three times. Again the umbilical cord is sterilized with 70% ethanol for 45 seconds. The umbilical cord pieces are washed with DPBS for 4 times to remove the traces of ethanol. The umbilical cord is cut into the pieces of 2-3 ems length and stored in a saline solution.

According to one embodiment herein, the mesenchymal stein cells are isolated and propagated by the following steps: a 5 ml of saline is taken in a sterile petridish and the umbilical cord piece is placed in the saline solution. A slit is made in the umbilical cord piece to expose the Wharton's jelly. The umbilical cord vessels are removed in the saline solution and the Wharton's jelly is removed using the sharp sterile forceps and a scalpel. The Wharton's jelly is collected in a sterile 50 ml centrifuge tube. The collected Wharton's jelly is centrifuged at 1500 rpm for 15 minutes. The supernatant is discarded and the pellet is washed once with Dulbecco's Phosphate Buffered Saline (DPBS). The cells are strained using the cell strainer (100 μm) in a sterile 50 ml falcon tube (around 45-50 ml). The cells are centrifuged at 1500 rpm for 15 minutes. The supernatant is discarded and the pellet is dissolved in a fresh culture medium. The cell count is done using a Haemocytometer. The cells are seeded on one cell stack with a density of 10000 cells per cm². A 100 ml of culture media supplemented with 1 ng/ml basic fibroblast growth factor (bFGF) is added to one chamber cell stack. The cell stack is gently rocked for an even distribution of the cells and labeled as “Passage 0” (P-0), with ANSA code and date. The cell stack is incubated at 37° C. with 5% CO₂ and is observed every alternate day. A complete media change is done after 72 hours of isolation procedure. After every 5 days, the spent media is partially replaced with a fresh culture medium. The cells are cultured for 15-20 days until the confluency of 80%-90% is achieved in the flask.

The mesenchymal stem cells have CD73, CD90 and CD166 antigens. The cells should be positive for these antigens. Further the mesenchymal stem cells should be negative for CD34 and CD48. As these antigens are expressed by hematopoietic stem cells, the hematopoietic stem cells are not mesenchymal stem cells in nature. Further to call a population of cells as mesenchymal stromal cells, more than 95% of the cells should be positive for CD73, CD90 and CD 166 antigens. Also the cells showing CD34 and CD48 antigens should not exceed 2%.

According to one embodiment herein, the characterization of the isolated and harvested mesenchymal stem cells from Wharton's jelly is carried out. The characterization process includes identification of the morphology i.e. spindle shape of the cells; immunophenotype identification which includes CD34−ve, CD45−ve, CD73+ve CD90±ve CD166+ve testing; and tri-lineage differentiation potential testing which includes testing for adipocyte, osteocyte and chondrocyte differentiation.

According to one embodiment herein, the characterized Wharton's jelly derived mesenchymal stem cells (NJ-MSC) are cryopreserved.

According to one embodiment herein, the Wharton's jelly derived mesenchymal stern cells (WJ-MSC) are injected to the glioblastoma patient directly to the tumor site. It is found that the tumors which do not respond to Temozolomide (TMZ) respond to WJ-MSC. Also WJ-MSC does not exert any toxic effect on any organ of the human body. WJ-MSC are cryoprotective to healthy cells and cytotoxic to glioblastoma cells.

According to one embodiment herein, Ocoprint® an explants tumor culture model to identify the anti-tumor effect of commonly used chemotherapeutics agents as well as Wharton's jelly derived mesenchymal stem cells on glioma's samples. In the laboratory, a tumor microenvironment similar to the body or physiological environment is created artificially in a dish. The tumor sample collected from the patient is placed in this micro-environment in dish. Different anticancer agents including the mesenchymal stem cells derived from umbilical cord are applied on the tumor in the micro-environment. This mimics the application of these anti-cancer agents against the tumor when the tumor is in the human body. Simultaneously a control sample is maintained without any treatment or application of anti-cancer agents. After a specific treatment time or incubation, the tumor samples are collected from the microenvironment and are subjected to a set of laboratory tests. The tests demonstrate the effect of anti-cancer agents on the tumor. The tests also reveal that the anti-cancer agents are able to kill tumor cells, reduce the number of tumor cells or limit the growth of tumor cells or the anti-cancer agent is converting the aggressively growing tumor cells to a programmed cell death mode.

The tests which are applied for testing effect of anti-cancer agents on the tumor are 1) Hematoxylin and eosin staining (H & E staining), 2) Ki67 is staining and 3) Caspase staining.

The H & F staining reveals the number of tumor cells present in the sample. The Ki67 staining indicates the growth pattern of tumor cells. The caspase staining shows that the aggressively growing cells turned into a programmed cell death mode or the dying cells.

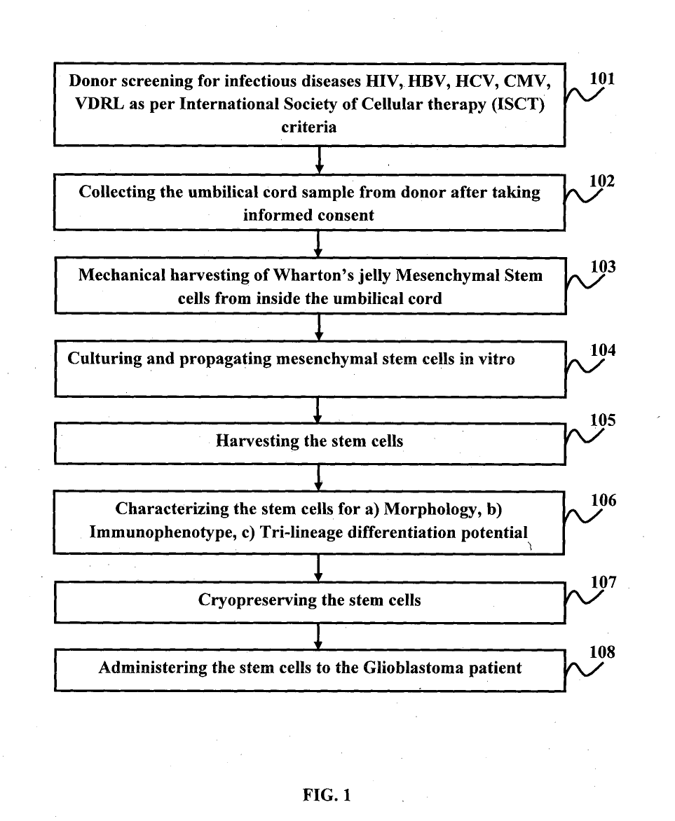

FIG. 1 shows a flow chart illustrating a method for treating glioblastoma patients from human umbilical cord derived Wharton jelly-mesenchymal stem cells, according to one embodiment herein. The method of treatment is initiated by screening donor for the infectious diseases HIV, HBV HCV, CMV, and VDRL as per International Society of Cellular therapy-ISCT criteria (101). A consent from the donor is taken for the collection of the umbilical cord sample and the donor is informed accordingly (102). The Wharton's jelly- mesenchymal stem cells are mechanically harvested from the umbilical cord (103). The isolated mesenchymal stem cells are cultured and propagated in vitro (104). The mesenchymal stem cells are harvested (105). The isolated and harvested mesenchymal stem cells are characterized (106). The mesenchymal stem cells are characterized for (a) morphology, (b) immunophenotype and (c) tri-lineage differentiation potential. The morphology of stem cells is spindle shaped. In the immunophenotype the CD34−ve, CD48−ve, CD73±ve, CD90±90, and CD166+ve are tested. The tri-lineage differentiation potential is tested to confirm that the mesenchymal stem cells differentiate into adipocytes, osteocytes and chondrocytes. The isolated and harvested Wharton's jelly-mesenchymal stem cells (WJ-MSC) are cryopreserved (107). The Wharton's jelly-mesenchymal stem cells (WJ-MSC) are administered to the glioblastoma patient. (108).

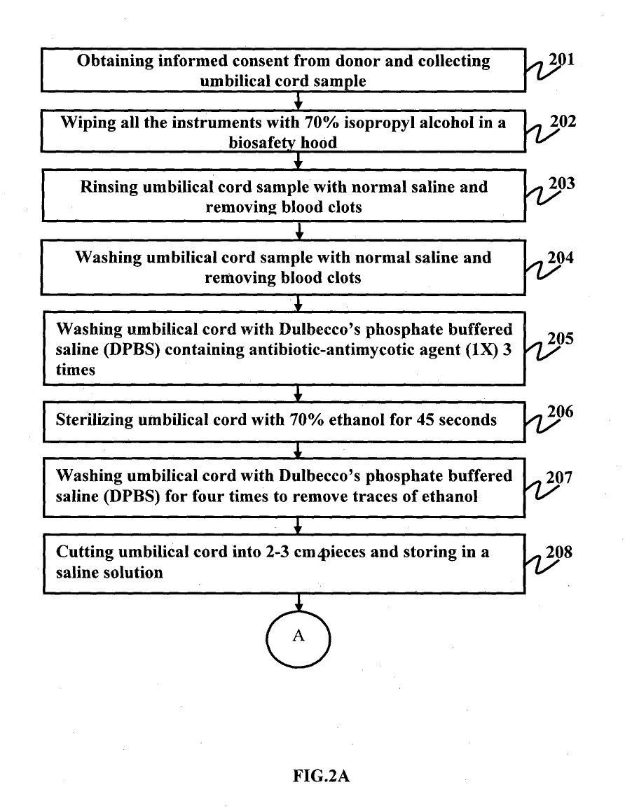

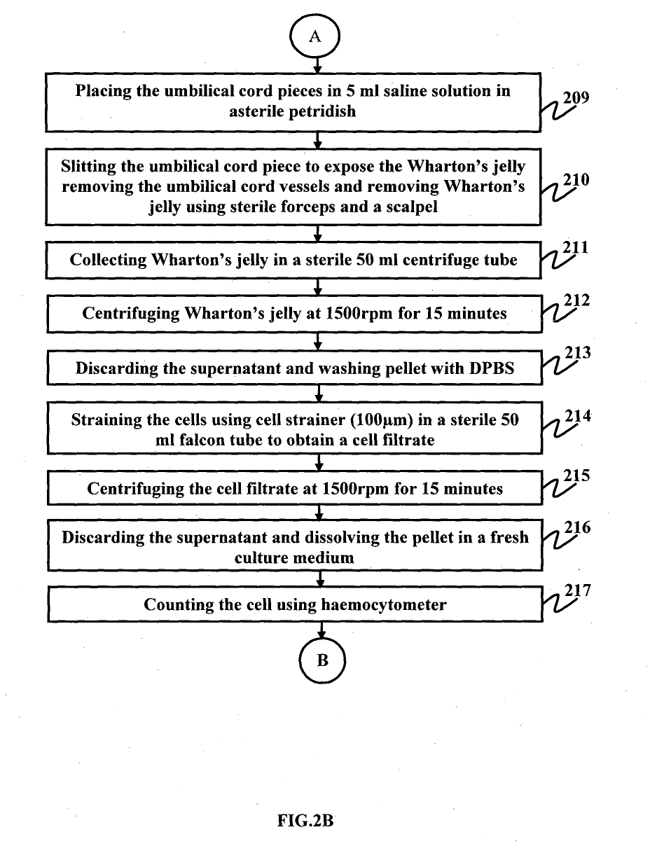

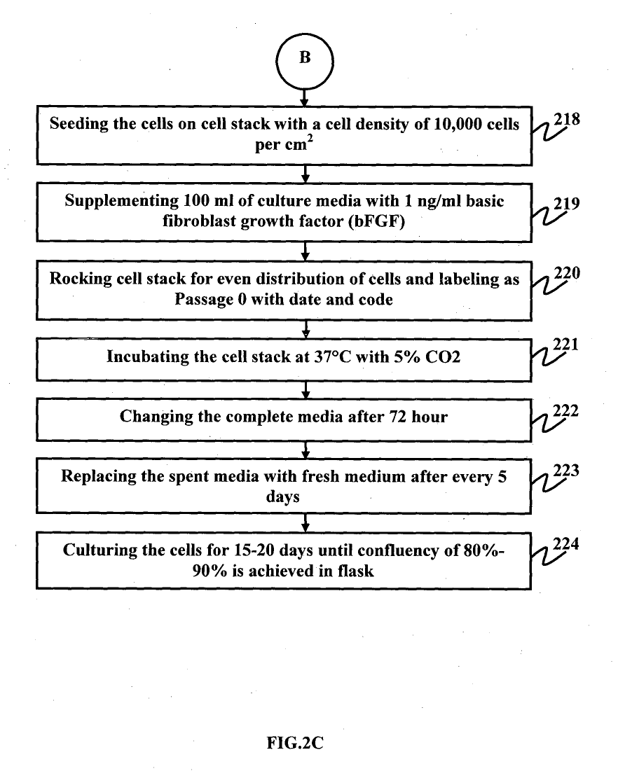

FIG. 2 shows a flow chart illustrating a method for isolation and maintenance of Wharton jelly-mesenchymal stem cells from human umbilical cord sample, according to one embodiment herein. The method of isolation is initiated by obtaining the consent from donor and collecting the umbilical cord sample. The donor is informed of the collection of the umbilical cord sample (201). The instruments are wiped with 70% isopropyl alcohol in a bio-safety hood (202). The umbilical cord sample is rinsed with normal saline and the blood clots are removed (203). The umbilical cord sample is washed with Dulbecco's phosphate buffered saline (DPBS) containing antibiotic-antimycotic agent (1×) for three times (204). The umbilical cord is again sterilized with 70% ethanol for 45 seconds (205). The umbilical cord sample is placed in the ethanol for 45 seconds. The umbilical cord is washed with DPBS for four times to remove the traces of ethanol (206). The umbilical cord is cut into several pieces with a length of 2-3 cm and the pieces are stored in a saline solution (207). The umbilical cord pieces are placed in a sterile petridish containing 5 ml saline solution (208). The umbilical cord pieces are slit to expose the Wharton's jelly (209). The umbilical cord vessels are removed using sharp sterile forceps and a scalpel (210). The Wharton's jelly is collected in a sterile 50 ml centrifuge tube (211). The Wharton's jelly is centrifuged at 15000 rpm for 15 minutes (212). The supernatant is discarded and the pellet is washed with DPBS (213). The cells are strained using cell strainer (100 μm) in a sterile 50 ml falcon tube to obtain a cell filtrate (214). The cell filtrate is centrifuged at 15000 rpm for 15 minutes (215). The supernatant is discarded and the pellet is dissolved in a fresh culture medium (216). The stem cells are counted using a Haemocytometer (217). The stem cells are seeded on the cell stack with a cell density of 10,000 cells per cm² (218). A 100 ml of culture media is supplemented with 1 ng/ml basic fibroblast growth factor (bFGF) (219). The cell stack is subjected to rocking for even distribution of cells and the cell stack is labeled as ‘Passage 0’ (P0) with ANSA code and date. The cell stack is incubated at 37° C. with 5% CO₂ (220). The complete media is replaced after 72 hours (221). The spent media is replaced with fresh medium after every 5 days (222). The cells are cultured for 15-20 days until confluency of 80%-90% is achieved in flask (723).

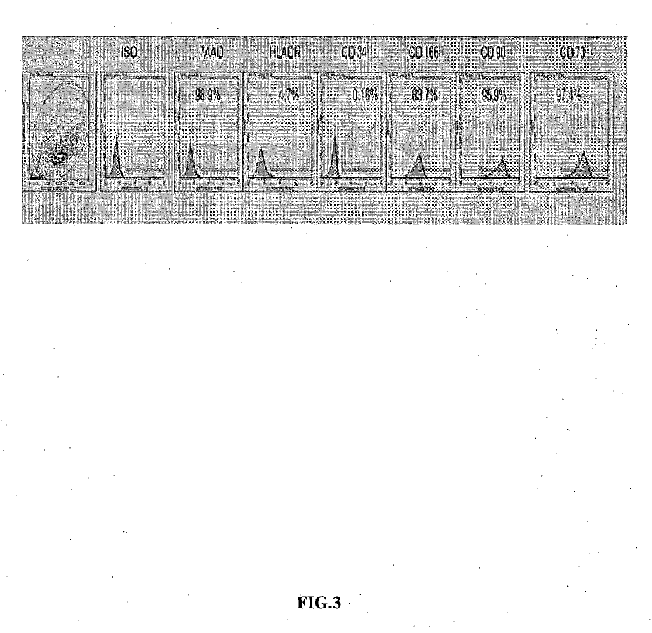

FIG. 3 illustrates a graph indicating the immunophenotype characterization of the Wharton jelly-mesenchymal stem cells, according to one embodiment herein. The graph reveals that the Wharton's jelly derived mesenchymal stem cells (WJ-MSC) are negative for CD34, CD48, and positive for CD73, CD90, and CD 166.

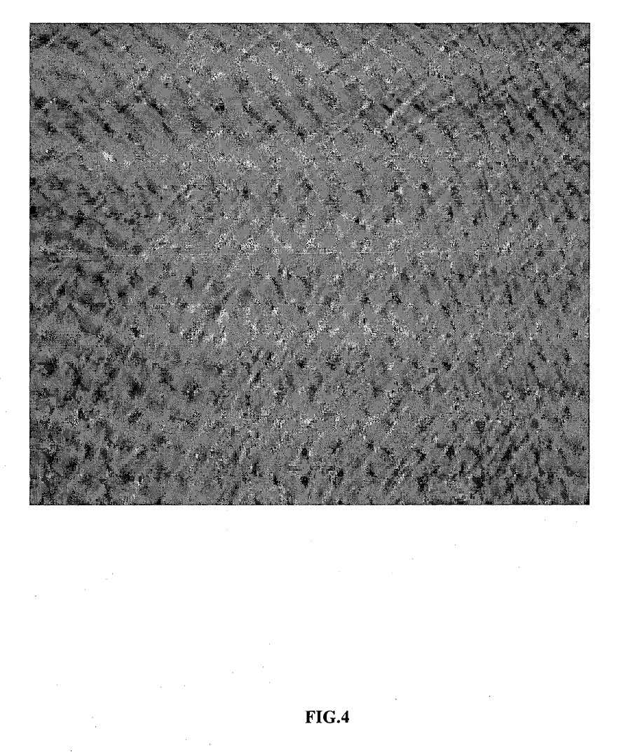

FIG. 4 illustrates a micrograph indicating the shape of mesenchymal stem cells isolated and cultured from human Wharton's jelly sample, according to one embodiment herein. The micrograph illustrates the spindle shape of the Wharton's jelly derived mesenchymal stem cells (WJ-MSC).

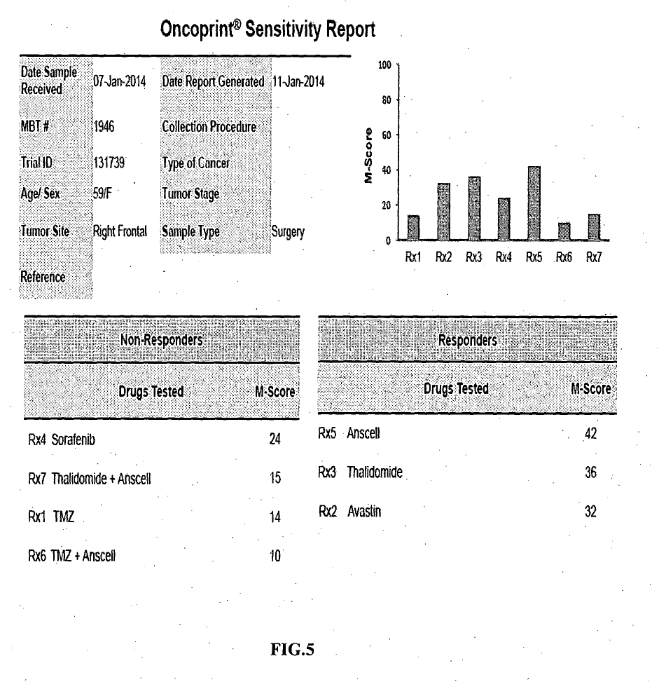

FIG. 5 illustrates an Oncoprint® sensitivity report indicating the drug sensitivity test, according to one embodiment herein. The graph in the report categorizes the drugs used (including cells) into two groups- responders and non-responders. The report illustrates that the glioma samples have responded to Wharton's jelly derived mesenchymal stem cells the most, followed by Thalidomide and Avastin. Also, the tumor cells in vitro have not responded to sorafenib, temzolamide or combination with cells.

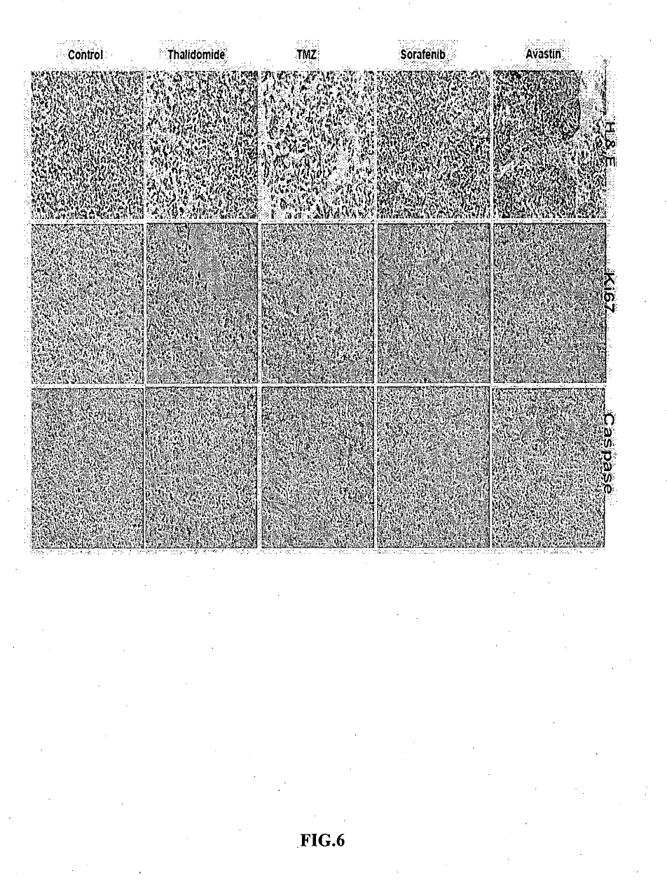

FIG. 6 illustrates a photograph indicating the effect of chemotherapeutic drugs on the tumor sample, according to one embodiment herein. The photograph illustrates the treatment of tumor cell with anti-cancer agents; specifically sorafenib thalidomide, avastin and temozolainide. After treating the tumor cells with the anti-cancer agents the tumor cells are subjected to hematoxylin and eosin staining (H & E staining), Ki67 is staining and caspase staining. The photograph further illustrates the H and E staining showing the infiltration of neutrophils into the tumour tissue, where the Ki67 measures the proliferation index and the Caspase is an immunohisthchemical marker for neoplasms.

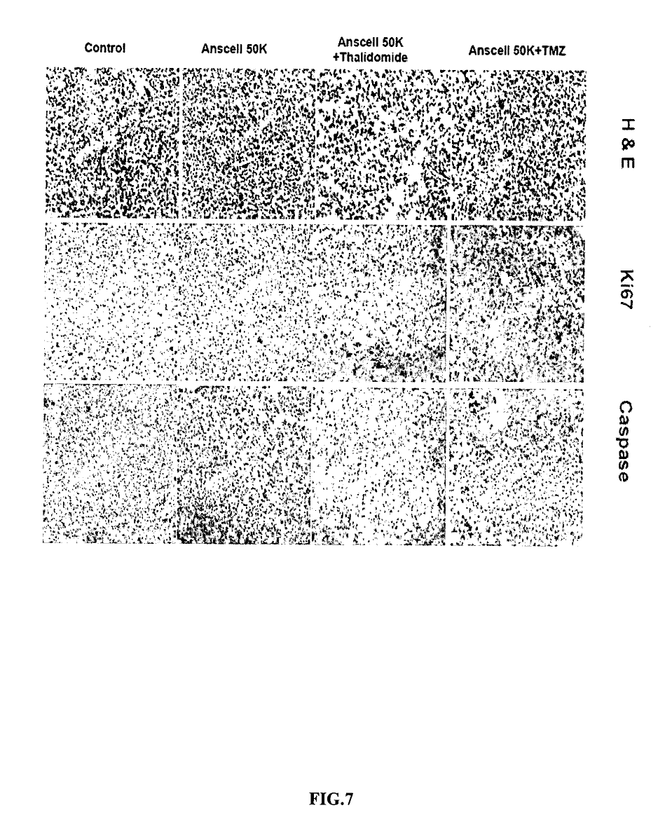

FIG. 7 illustrates a photograph indicating the effect of the mesenchymal stem cells on the tumors sample in vitro, according to one embodiment herein. The photograph illustrates the treatment of tumor cell with anti-cancer agents; specifically Anscell (mesenchymal stem cell derived from Wharton's jelly of umbilical cord), thalidomide+Anscell and Temozolamide+Anscell. After treating the tumor cells with the anti-cancer agents the tumor cells are subjected to hematoxylin and eosin staining (H&E staining), Ki67 is staining and caspase staining. The photograph illustrates that the glioma tumor samples have responded to Wharton's jelly derived mesenchymal stem cells.

The seven anti-cancer agents applied to the glioblastoma tumor are sorafenib, thalidomide, avastin, temozolamide, Anscell, Anscell+thalidone and Anscell+temozolamide. Among all the seven anti-cancer agents the Anscell (umbilical cord Wharton jelly derived mesenchymal stem cells) show the highest anti-tumor effect on gliooblastoma tumor cells. The H&E staining illustrates a comparative reduction in tumor cells. The Ki67 staining reveals that there is reduction in number of proliferating tumor cell and uncontrollable growth of cells. The caspase staining shows an increased number of apoptotic cells i.e. the tumor cells have turned from an aggressive mode of dying cells.

The foregoing description of the specific embodiments will so fully reveal the general nature of the embodiments herein that others can, by applying current knowledge, readily modify and/or adapt for various applications such specific embodiments without departing from the generic concept, and therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between

What is claimed is

1. A method for isolating mesenchymal stem cells from Wharton jelly of umbilical cord, the method comprising steps of:
collecting umbilical cord from a donor;
processing and preserving the umbilical cord;
isolating the mesenchymal stem cells from the Wharton jelly of umbilical cord;
analysing the isolated mesenchymal stem cells for surface antigens; and
cryopreserving the mesenchymal stem cells isolated from the Wharton jelly of umbilical cord.

2. The method according to claim 1, wherein the method of processing and preserving umbilical cord comprises the steps of:
obtaining informed consent from an umbilical cord donor and collecting umbilical cord;
wiping all instruments with alcohol in a biosafety hood, and wherein the alcohol is 70% v/v isopropyl alcohol;
rinsing the umbilical cord pieces with a saline solution and wherein the umbilical cord pieces are rinsed to remove blood clots;
washing the umbilical cord pieces with a Dulbecco's phosphate buffer saline solution, and wherein the buffer saline solution comprises an antibiotic, and wherein the antibiotic is an antimycotic agent (1×);
sterilizing the umbilical cord pieces with alcohol for 45 seconds, and
wherein the alcohol is 70% v/v ethanol;
washing the umbilical cord pieces with a Dulbecco's phosphate buffer saline (DBPS) for four times, and wherein the umbilical cord pieces are washed in DPBS to remove traces of ethanol;
cutting umbilical cord pieces into smaller pieces of 2-3 cm length; and
storing the umbilical cord pieces in a saline solution.

3. The method according to claim 1, wherein the method of isolating the mesenchymal stem cells from the Wharton jelly of umbilical cord comprises the steps of:
placing the umbilical cord in a sterile petridish, and wherein the petridish comprises 5 ml of a saline solution;
slitting the umbilical cord to expose the Wharton's jelly, and wherein the umbilical cord is slit using a forsep and scalpel;
collecting the Wharton's jelly in a sterile 50 ml centrifuge tube;
centrifuging Wharton's jelly at 1500 rpm for 15 minutes;
discarding the supernatant and washing the pellet with Dulbecco's Phosphate Buffer Saline (DBPS) to obtain the mesenchymal stem cells;
straining the cell using a cell strainer in a sterile 50 ml falcon tube; and
wherein the pore size of cell strainer is 100 μm;
centrifuging the cells at 15000 rpm for 15 minutes;
discarding the supernatant and dissolving the pellet in a fresh culture medium, and wherein the pellet comprises mesenchymal stem cells;
counting the mesenchymal stem cells using, a Haemocytometer;
seeding the mesenchymal stem cells on one cell stack with a density of 10,000 cells per cm², and wherein the cell stack comprises 100 ml of culture media supplemented with 1 ng/ml basic fibroblast growth factor (bFGF):
gently rocking the cell stack for an evenly distribution of the cells on a laboratory rocker;
incubating the cell stack at 37° C. with 5% CO₂;
changing the media completely after 72 hours of mesenchymal stein cell isolation process;
partially replacing the spent culture medium with a flesh culture medium after every five days: and
culturing the mesenchymal stem cells for 15-20 days until a confluency of 80-90° is obtained.

4. The method according to claim 1, wherein the isolated mesenchymal stem cells are analyzed for antigens, and wherein the isolated mesenchymal stem cells are tested positive for CD 73, CD 90 and CD 166 antigens, and wherein the mesenchymal stem cells are tested negative for CD34 and CD48 antigens, and wherein 95% of the mesenchymal stem cells are tsted positive for CD 73, CD 90 and CD166 antigens.

5. A method of targeting glioblastoma with Wharton's Jelly mesenchymal stem cells (WJ-NISCs) derived from human umbilical cord, the method comprises the steps of:
administering the Wharton's Jelly mesenchymal stem cells (WJ-MSCs) to the glioblastoma tumour; and
analyzing an effect of Wharton's Jelly mesenchymal stem cells (WJ-MSCs) on glioblastoma tumour.

6. The method according to claim 5, wherein the Wharton's Jelly mesenchymal stem cells (WJ-MSCs) are injected to the glioblastoma tumour in the laboratory conditions.

7. The method according to claim 5, wherein the Wharton's Jelly mesenchymal stem cells (WJ-MSCs) stop the growth of glioblastoma tumour cells and induces programmed cell death (apoptosis).

8. The method according to claim 5, wherein the Wharton's Jelly mesenchymal stem cells (WJ-MSCs) reduce the proliferating glioblastoma tumour cell and reduce the uncontrollable growth of glioblastoma tumour cell.
